(19) United States
(12) Patent Application Publication
(10) Pub. No.: US 2010/0112039 A1
(43) Pub. Date: May 6, 2010

(54) METHODS OF TREATING OVARIAN CANCER

(75) Inventors: Thomas M. Estok, Carlsbad, CA (US); Sara Zaknoen, Carlsbad, CA (US); Charles M. Baum, San Diego, CA (US)

Correspondence Address: MERCK;PATENT DEPARTMENT (K-6-1, 1990), 2000 GALLOPING HILL ROAD, KENILWORTH, NJ, 07033-0530, US

(73) Assignee: SCHERING CORPORATION, Kenilworth, NJ (US)

(21) Appl. No.: 12/446,919

(22) PCT Filed: Oct. 23, 2007
(86) PCT No.: PCT/US2007/022450

§ 371 Date: Jan. 7, 2010

Related U.S. Application Data
(60) Provisional application No. 60/854,344, filed on Oct. 25, 2006.

(51) Int. Cl. A61K 9/127; A61K 31/4985; A61P 35/00

(52) U.S. Cl. 424/450; 514/249

(57) ABSTRACT

Disclosed are methods of treating ovarian cancer (e.g., epithelial ovarian cancer) using lonafarnib, a taxane (e.g., paclitaxel or docetaxel) and a platinum coordinator complex (e.g., carboplatin, cisplatin or oxaliplatin). Also disclosed are methods of treating ovarian cancer (e.g. epithelial ovarian cancer) using lonafarnib, paclitaxel and carboplatin. Also disclosed are methods of treating ovarian cancer using lonafarnib in combination with a liposomal doxorubicin.

### REFERENCE TO RELATED APPLICATION

This application claims the benefit of U.S. Provisional Application No. 60/854,344 filed Oct. 25, 2006.

### BACKGROUND

Ovarian cancer is a leading cause of gynecologic cancer deaths. The majority of ovarian cancers (>90%) are epithelial in origin and often these cancers have already progressed to an advanced stage at the time of diagnosis.

In view of the interest in treating ovarian cancer, a method of treating ovarian cancer would be a welcome contribution to the art. This invention provides such a contribution.

### SUMMARY OF THE INVENTION

This invention provides a method of treating ovarian cancer in a patient in need of such treatment, said treatment comprising administering lonafarnib (e.g., the Sarasar® brand of lonafarnib) in combination with:

(1) a taxane (such as, for example, paclitaxel or docetaxel), and a platinum coordinator complex (such as, for example, carboplatin, cisplatin or oxaliplatin); or

(2) a liposomal doxorubicin (e.g., the Caelyx® brand of pegylated liposomal doxorubicin, and Doxil® brand of liposomal doxorubicin).

Thus, this invention provides a method of treating ovarian cancer in a patient in need of such treatment, said treatment comprising administering lonafarnib (e.g., the Sarasar® brand of lonafarnib) in combination with a taxane (such as, for example, paclitaxel or docetaxel), and a platinum coordinator complex (such as, for example, carboplatin, cisplatin or oxaliplatin).

This invention also provides a method of treating ovarian cancer in a patient in need of such treatment, said treatment comprising administering lonafarnib (e.g., the Sarasar® brand of lonafarnib) in combination with a liposomal doxorubicin (e.g., the Caelyx® brand of pegylated liposomal doxorubicin, and Doxil® brand of liposomal doxorubicin).

### DETAILED DESCRIPTION OF THE INVENTION

As used herein, unless otherwise defined, “patient” includes both human and animals. A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat. “Mammal” means humans and other mammalian animals.

As used herein, unless otherwise defined, “at least one” or “one or more” means there is 1 to several (e.g., 1, or 1 to 2, or 1 to 3 or 1 to 4, and the like).

As used herein, unless otherwise defined, “effective amount” or “therapeutically effective amount” is meant to describe an amount of drug, compound or composition effective in producing the desired therapeutic, ameliorative, inhibitory or preventative effect.

This invention provides a method of treating ovarian cancer in a patient in need of such treatment, said treatment comprising administering an effective amount of lonafarnib (e.g., the Sarasar® brand of lonafarnib) in combination with an effective amount of a taxane selected from the group consisting of: paclitaxel (e.g., the Taxol® brand of paclitaxel) and docetaxel (e.g., the Taxotere® brand of docetaxel), and an effective amount of a platinum coordinator complex selected from the group consisting of: carboplatin, cisplatin and oxaliplatin (e.g., the Eloxatin® brand of oxaliplatin).

This invention provides a method of treating ovarian cancer in a patient in need of such treatment, said treatment comprising administering an effective amount of lonafarnib (e.g., the Sarasar® brand of lonafarnib) in combination with an effective amount of a liposomal doxorubicin (e.g., the Caelyx® brand of pegylated liposomal doxorubicin, and Doxil® brand of liposomal doxorubicin).

Epithelial ovarian cancer is an example of the ovarian cancer treated in the methods of this invention. Examples of ovarian cancer also include fallopian tube cancer and primary peritoneal cancer.

Thus, one embodiment of this invention is directed to a method of treating ovarian cancer in a patient in need of such treatment, said treatment comprising administering lonafarnib in combination with the Caelyx® brand of pegylated liposomal doxorubicin.

Another embodiment of this invention is directed to a method of treating ovarian cancer in a patient in need of such treatment, said treatment comprising administering lonafarnib in combination with the Doxil® brand of liposomal doxorubicin.

Generally, carboplatin and paclitaxel are used in the methods comprising the administration of a taxane and a platinum coordinator complex.

One embodiment of this invention is directed to a method of treating ovarian cancer (e.g., epithelial ovarian cancer) in a patient in need of such treatment, said treatment comprising administering lonafarnib in combination with paclitaxel (e.g., the Taxol® brand of paclitaxel), and carboplatin.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered in an amount of about 25 to about 200 mg PO BID (i.e., orally twice per day) for each day of the treatment cycle;

(b) said paclitaxel is administered in an amount of about 100 mg/m² to about 200 mg/m²; and

(c) said carboplatin is administered in an amount to provide an AUC (mg/ml×min) of about 4 to about 8 once during the treatment cycle.

The starting doses of the therapeutic agents (e.g., the lonafarnib, the liposomal doxorubicin, the taxane and the platinum coordinator complex, and usually the taxane and the platinum coordinator complex, or either the taxane or platinum coordinator complex) can be adjusted by the skilled clinician in response to toxicity side effects in the patient.

Usually, in the embodiments of this invention, more than one treatment cycle is administered, i.e., usually the treatment cycle is repeated. Thus, in one embodiment of this invention the treatment cycle is repeated up to six times (e.g., six times).

Generally, a treatment cycle for the methods comprising the administration of a taxane and a platinum coordinator complex is 21 days.

A treatment cycle for the methods comprising the administration of a liposomal doxorubicin is, for example, 21 days or 28 days.

In the methods of this invention, lonafarnib is usually started on day 1 of the treatment cycle and administered continuously throughout each treatment cycle. Lonafarnib can be administered at doses of 25 mg to 200 mg PO BID (i.e., orally, twice a day). Generally, lonafarnib is administered at dose of 100 mg PO BID continuously during the treatment cycles. Usually, the doses of lonafarnib are given 12 hours apart, and usually the doses of lonafarnib are given 12 hours apart with food.

Usually paclitaxel is given once on day 1 of each treatment cycle. Usually paclitaxel is given as an infusion, and usually as a about a 3 hour infusion. Usually paclitaxel is administered in an amount of about 100 mg/m² to about 200 mg/m² once during the treatment cycle. Paclitaxel is generally administered at a dose of about 175 mg/m². Thus, paclitaxel is generally administered as about a 3 hour infusion at a dose of 175 mg/m².

Usually, carboplatin, is given once on day 1 of each treatment cycle. Usually carboplatin is administered as an infusion, and usually as about a 30 minute infusion. Usually, carboplatin is administered in an amount to provide an AUC (mg/ml×min) of about 4 to about 8. Carboplatin is generally administered in an amount to provide an AUC of 5 (mg/ml×min). Thus, carboplatin is generally administered as about a 30 minute infusion at a dose to provide and AUC of 5 (mg/ml×min).

Generally the infusion of paclitaxel is given first followed by the infusion of carboplatin.

Thus, another embodiment of this invention is directed to a method of treating ovarian cancer in a patient in need of such treatment, said treatment comprising administering lonafarnib in combination with paclitaxel and carboplatin, wherein:

(a) said lonafarnib is administered in an amount of about 100 mg twice per day on each day of the treatment cycle (i.e., lonafarnib is administered continuously during the treatment cycle);

(b) said paclitaxel is administered in an amount of about 175 mg/m² once during the treatment cycle; and

(c) said carboplatin is administered in an amount to provide an AUC (mg/ml×min) of about 5 once during the treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered in an amount of about 100 mg twice per day on each day of the treatment cycle (i.e., lonafarnib is administered continuously during the treatment cycle);

(b) said paclitaxel is administered in an amount of about 175 mg/m² once on day 1 of each treatment cycle; and

(c) said carboplatin is administered in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered in an amount of about 100 mg twice per day on each day of the treatment cycle (i.e., lonafarnib is administered continuously during the treatment cycle), wherein each dose is administered about 12 hours apart from the previous dose;

(b) said paclitaxel is administered as about a 3 hour infusion in an amount of about 175 mg/m² once on day 1 of each treatment cycle; and

(c) said carboplatin is administered as a 30 minute infusion in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered in an amount of about 100 mg twice per day on each day of the treatment cycle (i.e., lonafarnib is administered continuously during the treatment cycle), wherein each dose is administered about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered in an amount of about 100 mg twice per day on each day of the treatment cycle (i.e., lonafarnib is administered continuously during the treatment cycle), wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle); and

(b) said liposomal doxorubicin (e.g., the Caelyx® brand of pegylated liposomal doxorubicin) is administered in an amount of about 35 mg/m² to about 50 mg/m² (and in one example 45 mg/m² and in another example 50 mg/m²) on day 1 of a 28 day treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart; and

(b) said liposomal doxorubicin (e.g., the Caelyx® brand of pegylated liposomal doxorubicin) is administered in an amount of about 35 mg/m² to about 50 mg/m² (and in one example 45 mg/m² and in another example 50 mg/m²) on day 1 of a 28 day treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart with food; and

(b) said liposomal doxorubicin (e.g., the Caelyx® brand of pegylated liposomal doxorubicin) is administered in an amount of about 35 mg/m² to about 50 mg/m² (and in one example 45 mg/m² and in another example 50 mg/m²) on day 1 of a 28 day treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle); and

(b) said liposomal doxorubicin (e.g., the Caelyx® brand of pegylated liposomal doxorubicin) is administered in an amount of about 20 mg/m² once per week in a 3 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart; and

(b) said liposomal doxorubicin (e.g., the Caelyx® brand of pegylated liposomal doxorubicin) is administered in an amount of about 20 mg/m² once per week in a 3 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart with food; and

(b) said liposomal doxorubicin (e.g., the Caelyx® brand of pegylated liposomal doxorubicin) is administered in an amount of about 20 mg/m² once per week in a 3 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle); and

(b) said liposomal doxorubicin (e.g., the Caelyx® brand of pegylated liposomal doxorubicin) is administered in an amount of about 20 mg/m² once per week in a 4 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart; and

(b) said liposomal doxorubicin (e.g., the Caelyx® brand of pegylated liposomal doxorubicin) is administered in an amount of about 20 mg/m² once per week in a 4 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart with food; and

(b) said liposomal doxorubicin (e.g., the Caelyx® brand of pegylated liposomal doxorubicin) is administered in an amount of about 20 mg/m² once per week in a 4 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle); and

(b) said liposomal doxorubicin (e.g., the Doxil® brand of liposomal doxorubicin) is administered in an amount of about 50 mg/m² once during a 4 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart; and

(b) said liposomal doxorubicin (e.g., the Doxil® brand of liposomal doxorubicin) is administered in an amount of about 50 mg/m² once during a 4 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart with food; and

(b) said liposomal doxorubicin (e.g., the Doxil® brand of liposomal doxorubicin) is administered in an amount of about 50 mg/m² once during a 4 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle); and

(b) said liposomal doxorubicin (e.g., the Doxil® brand of liposomal doxorubicin) is administered in an amount of about 50 mg/m² once during a 3 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart; and

(b) said liposomal doxorubicin (e.g., the Doxil® brand of liposomal doxorubicin) is administered in an amount of about 50 mg/m² once during a 3 week treatment cycle.

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose of said lonafarnib is administered 12 hours apart with food; and

(b) said liposomal doxorubicin (e.g., the Doxil® brand of liposomal doxorubicin) is administered in an amount of about 50 mg/m² once during a 3 week treatment cycle.

The liposomal doxorubicin (e.g., the Caelyx® brand of pegylated liposomal doxorubicin) is usually administered IV over a one hour time period. The liposomal doxorubicin (e.g., the Caelyx® brand of pegylated liposomal doxorubicin) is usually administered for up to 6 treatment cycles.

The liposomal doxorubicin (e.g., the Doxil® brand of liposomal doxorubicin) is usually infused over a one hour time period. The liposomal doxorubicin (e.g., the Doxil® brand of liposomal doxorubicin) is usually administered for up to 6 treatment cycles (e.g., 3 to 6 treatment cycles).

Another embodiment of this invention is directed to any one of the embodiments described above wherein at the end of the treatment cycles lonafarnib is administered as a monotherapy. In the monotherapy Lonafarnib can be administered in an amount of about 25 to about 200 mg PO BID. Usually, lonafarnib is administered at a dose of 200 mg PO BID. Usually each dose is administered 12 hours apart, and usually each dose is administered with food.

The monotherapy with lonafarnib can be continued until disease progression or untoward toxicity.

Thus, the lonifarnib monotherapy can be continued as long as the patient experiences stable disease, NED (no evidence of disease) or objective response (CR/PR), and manageable toxicity.

In one example, the lonafarnib monotherapy can be continued for up to about 6 months (in one example, about 6 months) after cessation of the combination therapy (e.g., after cessation of the therapy with the taxane and the platinum coordinator complex).

Thus, for example, another embodiment of this invention is directed to a method of treating ovarian cancer in a patient in need of such treatment, said treatment comprising administering lonafarnib in combination with paclitaxel and carboplatin, wherein:

(a) said lonafarnib is administered in an amount of about 100 mg twice per day on each day of the treatment cycle (i.e., lonafarnib is administered continuously during the treatment cycle), wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle;

(d) said carboplatin is administered after said paclitaxel; and

(e) after the last treatment cycle (i.e, after cessation of combination therapy with paclitaxel and carboplatin), said lonafarnib is administered continuously as a monotherapy at a dose of about 200 mg PO BID (usually each dose being administered 12 hours apart, and usually each dose being administered with food).

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose is administered with food about 12 hours apart from the previous dose;

(b) said liposomal doxorubicin (e.g., the Caelyx® brand of pegylated liposomal doxorubicin) is administered in an amount of about 35 mg/m² to about 50 mg/m² (and in one example 45 mg/m² and in another example 50 mg/m²) on day 1 of a 28 day treatment cycle; and

(c) after the last treatment cycle (i.e, after cessation of combination therapy with the doxorubicin), said lonafarnib is administered continuously as a monotherapy at a dose of about 200 mg PO BID (usually each dose being administered 12 hours apart, and usually each dose being administered with food).

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose is administered with food about 12 hours apart from the previous dose;

(b) said liposomal doxorubicin (e.g., the Caelyx® brand of pegylated liposomal doxorubicin) is administered in an amount of about 20 mg/m² once per week in a 3 week treatment cycle; and

(c) after the last treatment cycle (i.e, after cessation of combination therapy with the doxorubicin), said lonafarnib is administered continuously as a monotherapy at a dose of about 200 mg PO BID (usually each dose being administered 12 hours apart, and usually each dose being administered with food).

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose is administered with food about 12 hours apart from the previous dose;

(b) said liposomal doxorubicin (e.g., the Caelyx® brand of pegylated liposomal doxorubicin) is administered in an amount of about 20 mg/m² once per week in a 4 week treatment cycle; and

(c) after the last treatment cycle (i.e, after cessation of combination therapy with the doxorubicin), said lonafarnib is administered continuously as a monotherapy at a dose of about 200 mg PO BID (usually each dose being administered 12 hours apart, and usually each dose being administered with food).

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose is administered with food about 12 hours apart from the previous dose;

(b) said liposomal doxorubicin (e.g., the Doxil® brand of liposomal doxorubicin) is administered in an amount of about 50 mg/m² once during a 4 week treatment cycle; and

(c) after the last treatment cycle (i.e, after cessation of combination therapy with the doxorubicin), said lonafarnib is administered continuously as a monotherapy at a dose of about 200 mg PO BID (usually each dose being administered 12 hours apart, and usually each dose being administered with food).

Another embodiment of this invention is directed to a method of treating ovarian cancer in a patient in need of such treatment, said treatment comprising administering lonafarnib in combination with a liposomal doxorubicin, wherein:

(a) said lonafarnib is administered in an amount of about 100 mg twice per day on each day of the treatment cycle (i.e., lonafarnib is administered continuously during the treatment cycle), wherein each dose is administered with food about 12 hours apart from the previous dose;

(b) said liposomal doxorubicin (e.g., the Doxil® brand of liposomal doxorubicin) is administered in an amount of about 50 mg/m² once during a 3 week treatment cycle; and

(c) after the last treatment cycle (i.e, after cessation of combination therapy with the doxorubicin), said lonafarnib is administered continuously as a monotherapy at a dose of about 200 mg PO BID (usually each dose being administered 12 hours apart, and usually each dose being administered with food).

In one example of the embodiments wherein lonofarnib monotherapy is administered, the lonafarnib monotherapy is continued until disease progression or untoward toxicity.

In one example of the embodiments wherein lonofarnib monotherapy is administered, the lonifarnib monotherapy is continued for as long as the patient experiences stable disease, NED (no evidence of disease) or objective response (CR/PR), and manageable toxicity.

In one example of the embodiments wherein lonofarnib monotherapy is administered, the lonafarnib monotherapy is continued for up to about 6 months (e.g., about 6 months).

Another embodiment of this invention is directed to any one of the embodiments described above wherein epithelial ovarian cancer is being treated.

In the methods of this invention wherein a platinum coordinator complex is administered, carboplatin is the preferred platinum coordinator complex used. Other platinum coordinator complexes, such as, for example, cisplatin or oxaliplatin, can be used in place of carboplatin. For example, the chemotherapeutic agent cisplatin can be used in an amount of about 30 mg/m² to about 100 mg/m² (for example, 75 mg/m²). Also, for example, the chemotherapeutic agent oxaliplatin (Eloxatin® brand of oxaliplatin) can be administered in an amount of 50-100 mg/m².

In the methods of this invention wherein a taxane is administered, paclitaxel is the preferred taxane used. Other taxanes, such as, for example, docetaxel, can be used in place of paclitaxel. For example, docetaxel (e.g., the Taxotere® brand of docetaxel) can be administered in an amount of about 50 to about 100 mg/m² (for example, 75 mg/m²).

Lonafarnib (available from Schering-Plough Corporation as the Sarasar® brand of lonafarnib) is an FPT inhibitor having the formula:

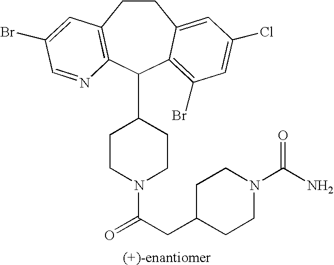

which compound can also be represented by the formula:

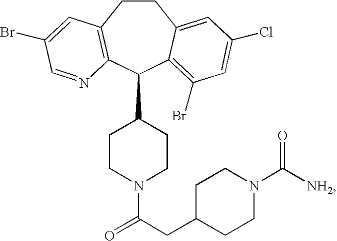

that is ((11R) 4[2[4-(3,10-dibromo-8-chloro-6,11-dihydro-5H-benzo[5,6]cyclohepta-[1,2-b]pyridin-11yl)-1-piperazinyl]-2-oxoethyl]-1-piperidinecarboxamide)). This compound is described in U.S. Pat. No. 5,874,442 issued Feb. 23, 1999, and WO99/32118 published Jul. 1, 1999, the disclosures of which are incorporated herein by reference thereto.

The therapeutic agents (e.g., paclitaxel and carboplatin) can be administered according to therapeutic protocols well known in the art for the treatment of ovarian cancer. Also, in accordance with the knowledge of the skilled clinician, the therapeutic protocols (e.g., dosage amounts and times of administration) can be varied in view of the observed effects of the administered therapeutic agents (e.g., lonafarnib, paclitaxel and carboplatin) on the patient, and in view of the observed responses of the ovarian cancer to the administered therapeutic agents.

The initial administration can be made according to established protocols known in the art, and then, based upon the observed effects, the dosage, modes of administration and times of administration can be modified by the skilled clinician.

Thus, in accordance with experience and knowledge, the practicing physician can modify each protocol for the administration of the therapeutic agents according to the individual patient's needs, as the treatment proceeds. All such modifications are within the scope of the present invention.

For example, the attending clinician, in judging whether treatment is effective at the dosage administered, will consider the general well-being of the patient as well as more definite signs such as relief of cancer-related symptoms (e.g., pain), inhibition of tumor growth, actual shrinkage of the tumor, or inhibition of metastasis. Size of the tumor, or tumor burden, can be measured by standard methods such as sequential measurements of serum CA-125 level or radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Response and progression to treatment can be evaluated according to criteria known in the art, such as the criteria proposed by the RECIST (Response Evaluation Criteria in Solid Tumors) committee (see, Therasse P, Arbuck S G, Eisenhauer E A et al., J Natl Cancer Inst 2000, 92:205-216, New guidelines to evaluate the response to treatment in solid tumors) with supplemental definitions of progression as published by Vergote et al., J Natl Cancer Inst 2000, 92:1534-1535, Re: new guidelines to evaluate the response to treatment in solid tumors [ovarian cancer]. Gynecological Cancer Intergroup.).

Progression-recurrence is defined according to the RECIST criteria and GCIG modifications and includes also: (a) occurrence (clinically or imaging signs) of any new lesion, (b) health status deterioration attributable to the disease, (c) death of any cause before progression is diagnosed, (d) CA 125 elevation as defined by the GCIG criteria, and (e) increase in measurable and/or non-measurable tumor as defined by the RECIST criteria.

Measurable disease can be defined as lesions that can accurately be measured in at least one dimension (longest diameter (LD) to be recorded) as a 20 mm with conventional techniques (or as a 10 mm with spiral CT scan). Nonmeasurable lesions can be defined as, all other lesions, including small lesions (LD<20 mm with conventional techniques or <10 mm with spiral CT scan) and truly nonmeasurable lesions. Lesions that are considered as truly nonmeasurable include: bone lesions, leptomeningeal disease, ascites, pleuraVpericardial effusion, inflammatory breast disease, lymphangitis cutis/putmonis, abdominal masses that are not confirmed and followed by imaging techniques, and cystic lesions. Measurable disease is the presence of at least one measurable lesion. If the measurable disease is restricted to a solitary lesion, its neoplastic nature should be confirmed by cytology/histology (techniques well known to those skilled in the art).

All measurable lesions up to a maximum of 5 lesions per organ and 10 lesions in total representative of all involved organs should be identifies as target lesions and recorded and measured at baseline. Target lesions should be selected on the basis of their size (lesion with the longest dimension) and their suitability for accurate repetitive measurements by one consistent method of assessment (either by imaging techniques or clinically). A sum of the longest dimension (LD) for all target lesions should be calculated and reported as the baseline sum LD. The baseline sum LD will be used as reference to further characterize the objective tumor response of the measurable dimension of the disease.

All other lesions (or sites of disease) should be identified as non-target lesions and should also be recorded at baseline. Measurements of these lesions are not required, but they should be followed as “absent”.

In the evaluation of target lesions: (1) a complete response (CR) means the disappearance of all target lesions; (2) a partial response (PR) means at least a 30% decrease in the sum of the longest diameter (LD) of target lesions, taking as reference the baseline sum LD; (3) stable disease (SD) means neither sufficient shrinkage to qualify for PR nor sufficient increase to qualify for PD (progressive disease), taking as reference the smallest sum LD since the treatment started; and (4) progressive disease (PD) means at least 20% increase in the sum of the LD of target lesions, taking as reference the smallest sum LD recorded since the treatment started or the appearance of one or more new lesions.

In the evaluation of non-target lesions: (1) a complete response (CR) means disappearance of all non-target lesions and normalization of tumor marker; (2) incomplete response/stable disease (SD) means persistence of one or more non-target lesions(s) and/or the maintenance of tumor marker level above the normal limits; and (3) progressive disease (PD) means appearance of one or more new lesions and/or unequivocal progression of existing non-target lesions.

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria.

Carboplatin is the preferred platinum coordinator complex used in the methods of this invention. Other platinum coordinator complexes may be used in place of carboplatin. For example, cisplatin administered in an amount of about 30 mg/m² to about 100 mg/m² (for example, 75 mg/m²). Also, for example, Oxaliplatin (Eloxatin) administered in an amount of 50-100 mg/m².

Paclitaxel is the preferred taxane used in the methods of this invention. Other taxanes may be used in place of paclitaxel. For example, Docetaxel (e.g., the Taxotere® brand of docetaxel) is administered in an amount of about 50 to about 100 mg/m² (for example, 75 mg/m²).

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A method of treating ovarian cancer in a patient in need of such treatment comprising administering to said patient an effective amount of lonafarnib in combination with an effective amount of a taxane selected from the group consisting of paclitaxel and docetaxel, and an effective amount of a platinum coordinator complex selected from the group consisting of: carboplatin, cisplatin and oxaliplatin.

2. A method of treating ovarian cancer in a patient in need of such treatment comprising administering to said patient an effective amount of lonafarnib in combination with an effective amount of carboplatin, and an effective amount of paclitaxel.

3. The method of claim 2 wherein epithelial ovarian cancer is treated.

4. The method of claim 2 wherein said lonafarnib, carboplatin and paclitaxel are administered for more than one treatment cycle.

5. The method of claim 2 wherein the treatment cycle is repeated 6 times.

6. The method of claim 3 wherein the treatment cycle is repeated 6 times.

7. The method of claim 4 wherein each treatment cycle is 21 days.

8. The method of claim 5 wherein each treatment cycle is 21 days.

9. The method of claim 6 wherein each treatment cycle is 21 days.

10. The method of claim 5 wherein the administration of lonafarnib is continued for up to six months after the last treatment cycle.

11. The method of claim 6 wherein the administration of lonafarnib is continued for up to six months after the sixth treatment cycle.

12. The method of claim 10 wherein each treatment cycle is 21 days.

13. The method of claim 11 wherein each treatment cycle is 21 days.

14. The method of claim 4 wherein said lonafarnib is administered on each day of each treatment cycle.

15. The method of claim 4 wherein said carboplatin is administered once on day 1 of each treatment cycle.

16. The method of claim 4 wherein said paclitaxel is administered once on day 1 of each treatment cycle.

17. The method of claim 4 wherein: said lonafarnib is administered on each day of each treatment cycle, said carboplatin is administered once on day 1 of each treatment cycle, and said paclitaxel is administered once on day 1 of each treatment cycle.

18. The method of claim 17 wherein there are 6 treatment cycles.

19. The method of claim 18 wherein each treatment cycle is 21 days.

20. The method of claim 19 wherein epithelial ovarian cancer is treated.

21. The method of claim 20 wherein the administration of lonafarnib is continued for up to 6 months after the last treatment cycle.

22. The method of claim 1 wherein:
(a) said lonafarnib is administered in an amount of about 25 to about 200 mg PO BID for each day of the treatment cycle;
(b) said paclitaxel is administered in an amount of about 100 mg/m² to about 200 mg/m² once during the treatment cycle; and
(c) said carboplatin is administered in an amount to provide an AUC (mg/ml×min) of about 4 to about 8 once during the treatment cycle.

23. The method of claim 22 wherein epithelial ovarian cancer is treated, and wherein there are 6 treatment cycles, and wherein each treatment cycle is 21 days.

24. The method of claim 23 wherein the administration of lonafarnib is continued for up to 6 months after the last treatment cycle.

25. The method of claim 1 wherein:
(a) said lonafarnib is administered in an amount of about 100 mg twice per day on each day of the treatment cycle;
(b) said paclitaxel is administered in an amount of about 175 mg/m² once during the treatment cycle; and
(c) said carboplatin is administered in an amount to provide an AUC (mg/ml×min) of about 5 once during the treatment cycle.

26. The method of claim 25 wherein epithelial ovarian cancer is treated, and wherein there are 6 treatment cycles, and wherein each treatment cycle is 21 days.

27. The method of claim 26 wherein the administration of lonafarnib is continued for up to 6 months after the last treatment cycle.

28. The method of claim 1 wherein:
(a) said lonafarnib is administered in an amount of about 100 mg twice per day on each day of the treatment cycle;
(b) said paclitaxel is administered in an amount of about 175 mg/m² once on day 1 of each treatment cycle; and
(c) said carboplatin is administered in an amount to provide an AUC (mg/ml×min) of about 5 once on day 1 of each treatment cycle.

29. The method of claim 28 wherein epithelial ovarian cancer is treated, and wherein there are 6 treatment cycles, and wherein each treatment cycle is 21 days.

30. The method of claim 29 wherein the administration of lonafarnib is continued for up to 6 months after the last treatment cycle.

31. The method of claim 1 wherein:
(a) said lonafarnib is administered in an amount of about 100 mg twice per day on each day of the treatment cycle, wherein each dose is administered about 12 hours apart from the previous dose;
(b) said paclitaxel is administered as about a 3 hour infusion in an amount of about 175 mg/m² once on day 1 of each treatment cycle; and
(c) said carboplatin is administered as a 30 minute infusion in an amount to provide an AUC (mg/ml×min) of about 5 once on day 1 of each treatment cycle.

32. The method of claim 31 wherein epithelial ovarian cancer is treated, and wherein there are 6 treatment cycles, and wherein each treatment cycle is 21 days.

33. The method of claim 32 wherein the administration of lonafarnib is continued for up to 6 months after the last treatment cycle.

34. The method of claim 1:
(a) said lonafarnib is administered in an amount of about 100 mg twice per day on each day of the treatment cycle, wherein each dose is administered about 12 hours apart from the previous dose;
(b) said paclitaxel is administered as about a 3 hour infusion in an amount of about 175 mg/m² once on day 1 of each treatment cycle;
(c) said carboplatin is administered as a 30 minute infusion in an amount to provide an AUC (mg/ml×min) of about 5 once on day 1 of each treatment cycle; and
(d) said carboplatin is administered after said paclitaxel.

35. The method of claim 34 wherein epithelial ovarian cancer is treated, and wherein there are 6 treatment cycles, and wherein each treatment cycle is 21 days.

36. The method of claim 35 wherein the administration of lonafarnib is continued for up to 6 months after the last treatment cycle.

37. The method of claim 1 wherein:
(a) said lonafarnib is administered in an amount of about 100 mg twice per day on each day of the treatment cycle, wherein each dose is administered with food about 12 hours apart from the previous dose;
(b) said paclitaxel is administered as about a 3 hour infusion in an amount of about 175 mg/m² once on day 1 of each treatment cycle;
(c) said carboplatin is administered as a 30 minute infusion in an amount to provide an AUC (mg/ml×min) of about 5 once on day 1 of each treatment cycle; and
(d) said carboplatin is administered after said paclitaxel.

38. The method of claim 37 wherein epithelial ovarian cancer is treated, and wherein there are 6 treatment cycles, and wherein each treatment cycle is 21 days.

39. The method of claim 1 wherein lonafarnib is administered as monotherapy after the last treatment cycle with said taxane and said platinum coordinator complex.

40. The method of claim 1 wherein lonafarnib is administered as monotherapy after the last treatment cycle with said taxane and said platinum coordinator complex, said monotherapy being continued until disease progression or untoward toxicity.

41. The method of claim 1 wherein lonafarnib is administered as monotherapy after the last treatment cycle with said taxane and said platinum coordinator complex, said monotherapy being continued until the patient experiences stable disease, no evidence of disease or objective response, and manageable toxicity.

42. The method of claim 37 wherein lonafarnib is administered as monotherapy after the last treatment cycle with said taxane and said platinum coordinator complex, said lonafarnib being administered in an amount of about 200 mg BID in said monotherapy.

43. The method of claim 37 wherein lonafarnib is administered as monotherapy after the last treatment cycle with said taxane and said platinum coordinator complex, said monotherapy being continued until disease progression or untoward toxicity, said lonafarnib being administered in an amount of about 200 mg BID in said monotherapy.

44. The method of claim 37 wherein lonafarnib is administered as monotherapy after the last treatment cycle with said taxane and said platinum coordinator complex, said monotherapy being continued until the patient experiences stable disease, no evidence of disease or objective response, and manageable toxicity, said lonafarnib being administered in an amount of about 200 mg BID in said monotherapy.

45. The method of claim 38 wherein the administration of lonafarnib is continued for up to 6 months after the last treatment cycle.

46. The method of claim 1 wherein:
(a) said lonafarnib is administered in an amount of about 100 mg twice per day on each day of the treatment cycle, wherein each dose is administered with food about 12 hours apart from the previous dose;
(b) said paclitaxel is administered as about a 3 hour infusion in an amount of about 175 mg/m² once on day 1 of each treatment cycle;
(c) said carboplatin is administered as a 30 minute infusion in an amount to provide an AUC (mg/ml min) of about 5 once on day 1 of each treatment cycle;
(d) said carboplatin is administered after said paclitaxel; and
(e) after the last treatment cycle, lonafarnib is administered continuously for up to 6 months at a dose of about 200 mg PO BID.

47. The method of claim 46 wherein each dose of said lonafarnib in (e) is administered 12 hours apart.

48. The method of claim 47 wherein each dose of lonafarnib in (e) is administered with food.

49. The method of claim 46 wherein epithelial ovarian cancer is treated, and wherein there are 6 treatment cycles, and wherein each treatment cycle is 21 days.

50. The method of claim 47 wherein epithelial ovarian cancer is treated, and wherein there are 6 treatment cycles, and wherein each treatment cycle is 21 days.

51. The method of claim 48 wherein epithelial ovarian cancer is treated, and wherein there are 6 treatment cycles, and wherein each treatment cycle is 21 days.

52. The method of claim 1 wherein said platinum complex coordinator is cisplatin.

53. The method of claim 1 wherein said platinum complex coordinator is oxalplatin.

54. The method of claim 1 wherein said taxane is docetaxel.

55. The method of claim 1 wherein the platinum complex coordinator is cisplatin, and said taxane is docetaxel.

56. A method of treating ovarian cancer in a patient in need of such treatment, said treatment comprising administering an effective amount of lonafarnib in combination with an effective amount of a liposomal doxorubicin.

57. The method of claim 56 wherein:
(a) said lonafarnib is administered in an amount of about 100 mg twice per day on each day of the treatment cycle; and
(b) said liposomal doxorubicin is the Caelyx® brand of pegylated liposomal doxorubicin administered in an amount of about 35 mg/m² to about 50 mg/m² on day 1 of a 28 day treatment cycle.

58. The method of claim 57 wherein each dose of said lonafarnib is administered 12 hours apart.

59. The method of claim 58 wherein each dose of said lonafarnib is administered with food.

60. The method of claim 57 wherein lonafarnib is administered as monotherapy after the last treatment cycle with said doxorubicin, said monotherapy being continued until disease progression or untoward toxicity, said lonafarnib being administered in an amount of about 200 mg BID in said monotherapy.

61. The method of claim 56 wherein:
(a) said lonafarnib is administered in an amount of about 100 mg twice per day on each day of the treatment cycle; and
(b) said liposomal doxorubicin is the Caelyx® brand of pegylated liposomal doxorubicin administered in an amount of about 20 mg/m² once per week in a 3 week treatment cycle.

62. The method of claim 61 wherein each close of said lonafarnib is administered 12 hours apart.

63. The method of claim 62 wherein each dose of said lonafarnib is administered with food.

64. The method of claim 61 wherein lonafarnib is administered as monotherapy after the last treatment cycle with said doxorubicin, said monotherapy being continued until disease progression or untoward toxicity, said lonafarnib being administered in an amount of about 200 mg BID in said monotherapy.

65. The method of claim 56 wherein:
(a) said lonafarnib is administered in an amount of about 100 mg twice per day on each day of the treatment cycle; and
(b) said liposomal doxorubicin is the Caelyx® brand of pegylated liposomal doxorubicin administered in an amount of about 20 mg/m² once per week in a 4 week treatment cycle.

66. The method of claim 65 wherein each dose of said lonafarnib is administered 12 hours apart.

67. The method of claim 66 wherein each dose of said lonafarnib is administered with food.

68. The method of claim 65 wherein lonafarnib is administered as monotherapy after the last treatment cycle with said doxorubicin, said monotherapy being continued until disease progression or untoward toxicity, said lonafarnib being administered in an amount of about 200 mg BID in said monotherapy.

69. The method of claim 56 wherein:
(a) said lonafarnib is administered in an amount of about 100 mg twice per day on each day of the treatment cycle; and
(b) said liposomal doxorubicin is the Doxil® brand of liposomal doxorubicin administered in an amount of about 50 mg/m² once during a 4 week treatment cycle.

70. The method of claim 69 wherein each dose of said lonafarnib is administered 12 hours apart.

71. The method of claim 70 wherein each dose of said lonafarnib is administered with food.

72. The method of claim 69 wherein lonafarnib is administered as monotherapy after the last treatment cycle with said doxorubicin, said monotherapy being continued until disease progression or untoward toxicity, said lonafarnib being administered in an amount of about 200 mg BID in said monotherapy.
